(19) United States
(12) Patent Application Publication
(10) Pub. No.: US 2019/0142321 A1
(43) Pub. Date: May 16, 2019

(54) DISPOSABLE PAINLESS LANCET AND LANCING DEVICE

(71) Applicants: Lim CheI CHOI, Busan (KR); Jeong Won CHOI, Gangwon-do (KR)

(72) Inventors: Lim Chel CHOI, Busan (KR); Jeong Won CHOI, Gangwon-do (KR); Min Gi CHOI, Busan (KR); Ho Im HA, Gangwon-do (KR)

(21) Appl. No.: 16/097,871

(22) PCT Filed: May 2, 2017
(86) PCT No.: PCT/KR2017/004661

§ 371 Date: Oct. 31, 2018

(30) Foreign Application Priority Data
May 2, 2016 (KR) 10-2016-0053935
Jul. 6, 2016 (KR) 10-2016-0085674
Mar. 22, 2017 (KR) 10-2017-0036096

(51) Int. Cl. A61B 5/151; A61B 5/15

(52) U.S. Cl. CPC A61B 5/15142; A61B 5/150633; A61B 5/150022; A61B 5/15115

(57) ABSTRACT

Provided is a lancing device including a casing, a launch part, and a bumper part wherein the bumper part comprises a needle body holder, a cylindrical bumper and bump spring; and a disposable painless lancet for use with the device. The disposable painless lancet and lancing device according to the present invention are figured such that a bump spring strikes the skin in advance before the needle penetrates the skin, disturbing the skin nerve, and such that a needle of the lancet can be allowed to be inserted only a certain depth of skin, thus the subject does not feel any pain at all.

### FIELD OF THE INVENTION

The present invention relates to disposable painless lancets and lancing devices for use therewith, which the subject can hardly feel to such an extent that a pain can be neglected. The lancets and the lancing devices of the present invention are configured such that a bump spring strikes a skin in advance before a needle penetrates the skin so as to disturb the skin nerve, and further configured such that, after the needle stabs the skin, the needle can be momentarily released out of the skin due to the restoring force of the bump spring so that a time required for the needle to stay in the skin is very short, and the needle can be adjusted to penetrate deep or shallow by allowing the needle to be inserted only a certain depth of skin.

### BACKGROUND OF THE INVENTION

A small amount of blood can be shed to remove coagulum and metabolic waste matter in oriental medicine. In addition, a small amount of blood can be also sampled to measure blood sugar, blood type, and other blood tests in western medicine. In this case, the subject can feel frightened by the pain of the lancet, i.e., the pain caused by the needle.

A human skin is generally divided into an epidermis (about 0.2 mm), a dermis (about 2 to 3 mm) and a subcutaneous tissue. The subcutaneous tissue has arteries and veins, and in the dermis, fine capillary blood vessels are distributed like a network. In order to sample blood, it is necessary to penetrate the needle up to the capillary blood vessels, that is, just before the subcutaneous tissue. Conventionally, as the needle stabs the skin, the needle can pass through the subcutaneous tissue and penetrate excessively too deeply, resulting in a severe pain. In order to solve such a problem, lancets having various lengths are commercially available, and the user has an inconvenience of using a lancet suitable for him as needed.

Korean Patent No. 10-1360939 discloses a lancet which does not penetrate the skin through a certain depth or more but merely draws blood, wherein the lancet has double bumps (multi-bumps) each having a cross-sectional area wider than that of a middle of the needle, thereby preventing the needle from being excessively deeply inserted. In the patent, the lancet is intended to reduce a length of the needle slightly so that the needle can penetrate slightly into the skin to reduce pain. Since a thickness of a human skin varies depending on a skin condition of a person, there is a disadvantage that a penetrating depth of the needle cannot be adjusted. In addition, even though the needle of the lancet is as short as possible, if the needle is directly inserted into the skin, the subject feels pain, so that a true painless effect cannot be obtained.

A complicated device using a projectile, a trigger, and a laser is known to prevent the needle from reaching a certain depth or more of the skin. However, since a disposable lancet is discarded after being used once, its price should be low and its structure should be simple.

Also, even if the lancet is disposable, there is always a risk that secondary infections such as AIDS and hepatitis can be infected unless special care is taken after use.

Prior arts related to a device for sampling blood include the following. For example, Korean Patent No. 10-0932946 discloses a device for securing a straight direction when a lancing needle penetrates the skin, but the device is not related to a function of eliminating a pain.

Korean Patent No. 10-0912202 discloses a lancet integrated cap and a painless lancet. The patent describes caps 210, 310, 510 for accommodating a lancet body 140, 220, 320, 520 as the striking means, similar to the present invention. However, since the caps as the striking means must be spaced apart from the skin to strike the skin, it is difficult for the user to maintain a proper distance. For example, if the distance between the skin and the cap is too far compared to the proper distance, the needle will not penetrate the skin. Conversely, if the distance between the skin and the cap is too close compared to the proper distance, there will be no striking effect. Since the striking strength and the depth of penetration of the needle vary according to the user, a consistency of a painless effect cannot be maintained. Therefore, there is a problem that a perfect painless effect cannot be expected in this device. In addition, since a lancet body and the cap are integrated, both the lancet body and the cap should be disposed of after use. Therefore, there is a problem in that the manufacturing cost is increased as well as a waste of resources.

### DETAILED DESCRIPTION OF THE INVENTION

### Problems to be Solved

As a result of researching to solve the above-mentioned problems, the present inventors have found: that the subject cannot feel pain when the needle is stuck by providing a coil-type bump spring embedded or fixed in the needle body so as to surround the needle and to protrude longer than the needle, since the bump spring strikes around the skin instantly before the needle of the lancet pierces the skin, thereby disturbing the skin nerve and allowing the needle to stay a very short time in the skin; that the problem of secondary infection due to careless handling after use can be solved, since the bump spring surrounds and protects the needle; and that it is possible to prevent the needle from excessively and deeply penetrating to below the subcutaneous tissue, since the needle penetrates the skin only as much as the length of the needle protruding from the bump spring when the bump spring is compressed by impact. The present invention has been completed based on these finding.

Therefore, an object of the present invention is to provide a simple and inexpensive disposable painless lancet and a device for use therewith, wherein the bump spring is provided with the needle body to disturb the skin nerve, the needle is allowed to penetrate only a certain depth of the skin, and the needle can be adjusted to pierce the skin deep or shallow, so that the subject can hardly feel the pain and the blood on the needle does not come into contact with the outside after use.

### Solution to the Problem

An aspect of the first embodiment of the present invention provides a painless lancing device.

The painless lancing device comprises a casing which includes a cylindrical front cap, a sleeve connected to the cylindrical front cap at a lower end and a rear cap connected to an upper end of the sleeve, a launch part disposed in the casing configured to launch a cylindrical bumper and a needle body to strike a skin. The bumper part is disposed in the casing and connected to the launch part by a coupling shaft.

Further, the bumper part includes a needle body holder which has a holder flange disposed at an upper portion thereof, a first rectangular hole disposed below the holder flange so that a stop bar moves up and down, and a needle body insertion hole disposed at a lower portion of the bumper part to insert a lancet. The cylindrical bumper is configured to receive the needle body holder at an upper end thereof, and further includes a second rectangular hole disposed along a longitudinal direction to insert and remove the lancet, a needle access hole disposed at a lower end of the cylindrical bumper for a needle, a needle spring to move in and out therethrough, and a stop bar insertion port disposed at the upper end to insert the stop bar. Further, the lancing device includes a bump spring surrounding a cylindrical body and disposed between a lower portion of the holder flange of the needle body holder and an upper portion of the cylindrical bumper.

Another aspect of the first embodiment of the present invention provides the painless lancing device, wherein one side of the needle body insertion hole is configured to allow insertion and removal of the needle body from said one side, and the needle body is disposed to align and the upper and lower surfaces of the needle body insertion hole are aligned with the upper and lower surfaces of the flat needle body.

Still another aspect of the first embodiment of the present invention provides the painless lancing device, wherein the cylindrical body has a lower end configured to allow the needle of the lancet and the needle spring to move in and out, wherein a ring-shaped protrusion is disposed at an inside surface of an upper end of the holder flange so that the ring-shaped protrusion is engaged with a circular-shaped coupling groove disposed along a lower outer peripheral surface of the coupling shaft, and wherein the holder flange is disposed on its outer circumferential surface thereof for screw coupling with a spiral disposed on an inside surface of a depth adjusting member.

Still another aspect of the first embodiment of the present invention provides the painless lancing device, wherein the upper and lower surfaces of the needle body insertion holes have needle body fixing protrusions for fixing the needle body.

Still another aspect of the first embodiment of the present invention provides the painless lancing device, wherein the lancet further includes a rectangular needle body having protrusions on both sides thereof, a needle fixing protrusion for inserting and fixing the needle springs on the protrusion, a needle inserted into the needle body through the needle fixing protrusion, and fixing grooves to be fixed on the needle body fixing protrusions provided on both inner walls of the needle body insertion hole.

Still another aspect of the first embodiment of the present invention provides the painless lancing device, which further includes a depth adjusting member configured to adjust a depth of skin penetration of the needle and having an open upper end and an open lower end, the depth adjusting member including a spiral disposed in an inner surface thereof for engaging with a screw thread disposed on an outer circumferential surface of the holder flange of the needle body holder, and an adjusting member flange disposed at a lower end of the depth adjusting member.

Still another aspect of the first embodiment of the present invention provides the painless lancing device, wherein the bumper part is connected to the coupling shaft by engaging a ring-shaped protrusion disposed at an inside surface of the holder flange with a circular coupling groove disposed along a lower portion of the coupling shaft, the coupling shaft having a coupling shaft flange for inserting a return spring therein.

An aspect of the second embodiment of the present invention provides a disposable painless lancet including a needle body, a needle embedded at the center of one end of the needle body, and a bump spring embedded or fixed at said one end of the needle body so as to surround the needle and extend beyond the needle.

An aspect of the second embodiment of the present invention provides the disposable painless lancet, wherein the needle body has an elongated spiral part disposed at one end thereof; wherein one end of the needle is embedded in the center of the end of the spiral part; wherein a spring body in the form of a nut having a spiral disposed therein so as to be screwed with the elongated spiral part is included; and wherein one end of the bump spring is embedded or fixed to the spring body.

Still another aspect of the second embodiment of the present invention provides the disposable painless lancet, wherein a silicone or elastic member is used instead of the bump spring.

Still another aspect of the second embodiment of the present invention provides a painless lancing device including: a casing including a cylindrical front cap, a sleeve having one side connected to the front cap and the other side connected to a rear cap which is connected with the sleeve; a launch part for launching a needle body disposed inside a cylindrical body; and a needle body holder for holding and fixing the needle body. The cylindrical front cap has a gradually tapering shape from a top end to a bottom end, and includes a circular engagement protrusion disposed on the inside of the front cap to limit the downward movement of the lower surface of the cylindrical body and a guide hole provided from the engagement protrusion to the bottom end of the front cap to guide the needle body and a bump spring in a predetermined direction. The needle body holder includes a ring-shaped protrusion disposed inside the needle body holder so as to be engaged with a coupling groove of the coupling shaft, and a needle body holder is open at its bottom portion and has one incision groove cut from the lower portion to a middle portion thereof.

### Effects of the Invention

The painless lancet and the lancing device to use therewith according to the present invention can control the needle to be inserted only to a certain depth of the skin without changing various sizes of the needle so that the needle does not penetrate below the subcutaneous tissues. The needle is instantly released out of the skin due to the restoring force of the bump spring after the needle has pierced the skin, and the time required for the needle to stay in the skin is shortened, and thus the subject does not feel any pain at all.

### BRIEF DESCRIPTION OF THE DRAWINGS

FIG. 1 is a perspective view of the lancing device according to a first embodiment of the present invention.

FIG. 2 is a perspective view of the lancing device according to the first embodiment of the present invention showing a state in which a front cap, a sleeve, and a rear cap, which form an external appearance, are disassembled.

FIG. 3 is a longitudinal sectional view of the lancing device according to the first embodiment of the present invention cut out at an angle of 90 degrees.

FIG. 4 is a longitudinal sectional view of the lancing device according to the first embodiment of the present invention.

FIG. 5 is a longitudinal sectional view of the lancing device according to the first embodiment of the present invention in which a front cap, a sleeve, and a rear cap are omitted.

FIG. 6 is a drawing showing an internal structure of the lancing device according to the first embodiment of the present invention.

FIG. 7 is a drawing showing an operating state of a launch part.

FIG. 8 is a cross-sectional view of a bumper part and a coupling shaft of the lancing device according to the first embodiment of the present invention, in which the bumper part is coupled with the coupling shaft.

FIG. 9 is a drawing showing a state in which a needle body holder and a depth adjusting member of the bumper part of the lancing device according to the first embodiment of the present invention are disassembled and the needle body holder and the coupling shaft are engaged.

FIG. 10 is an enlarged perspective view of the bumper part of the lancing device according to the first embodiment of the present invention.

FIG. 11 is an exploded perspective view of the bumper part of the lancing device according to the first embodiment of the present invention.

FIG. 12 is a drawing showing a state in which the depth adjusting member of the lancing device according to the first embodiment of the present invention is engaged with the needle body holder, and the needle body is inserted into a needle body hole or disassembled therefrom.

FIG. 13 is an enlarged longitudinal sectional view of the bumper part for showing an operating state of the bumper part of the lancing device according to the first embodiment of the present invention, in which FIG. 13 (a) illustrates a state of the lancet before launching, and FIG. 13 (b) illustrates a state in which the lancet penetrates the skin immediately after the lancet is launched.

FIG. 14 is a perspective view of a disposable painless lancet according to a second embodiment of the present invention, in which FIG. 14 (a) illustrates a state before use in which the bump spring is expanded, and FIG. 14 (b) illustrates a state in which the needle penetrates the skin and the bump spring is compressed.

FIG. 15 is another perspective view of a disposable painless lancet according to the second embodiment of the present invention.

FIG. 16 is a longitudinal sectional view of the disposable painless lancet according to the second embodiment of the present invention.

FIG. 17 is another perspective view of the disposable painless lancet according to the second embodiment of the present invention, in which the lancet is disassembled.

FIG. 18 is another perspective view of the disposable painless lancet according to the second embodiment of the present invention, in which a silicone or elastic member is used in place of the bump spring, in which FIG. 18 (a) illustrates a state before use in which the silicone or elastic member is expanded, and FIG. 18 (b) illustrates a state in which the needle penetrates the skin and the silicone or elastic member is compressed.

FIG. 19 is another perspective view of the lancing device according to the second embodiment of the present invention illustrating a state in which a front cap, a sleeve, and a rear cap, which form an external appearance, are disassembled.

FIG. 20 is another longitudinal sectional view of the lancing device according to the second embodiment of the present invention.

FIG. 21 is a drawing showing an internal structure of the lancing device according to the second embodiment of the present invention.

FIG. 22 is a cross-sectional view of a holder part and a coupling shaft of the lancing device according to the second embodiment of the present invention, in which the holder part is coupled with the coupling shaft, and the lancet is mounted to the device.

FIG. 23 is a drawing illustrating a structure in which the holder part is coupled with the coupling shaft, and the lancet is mounted to the device in accordance with the second embodiment of the present invention.

FIG. 24 is a drawing illustrating a state in which the rectangular part, the coupling shaft and the holder part of the lancing device according to a second embodiment of the present invention are assembled.

FIG. 25 is a drawing illustrating a state in which the needle body of FIG. 24 is disassembled.

FIG. 26 is a drawing illustrating the operation states of the bump spring and the needle of the lancing device according to the second embodiment of the present invention, in which FIG. 26(a) illustrates a state before launching and FIG. 26(b) illustrates a state after launching.

### THE BEST MODE FOR CARRYING OUT THE INVENTION

Hereinafter, the present invention will be described in more detail with reference to the accompanying drawings. However, the contents shown in the detailed description and in the drawings do not limit the present invention.

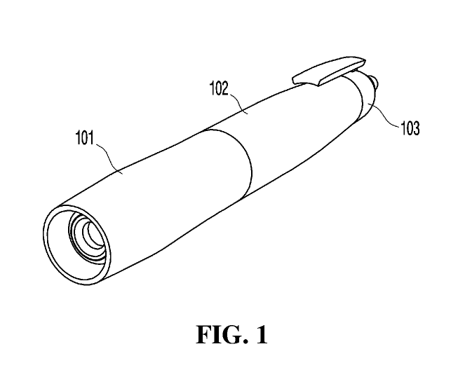

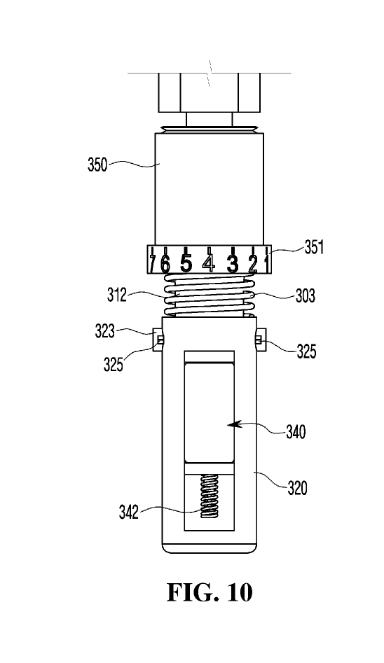

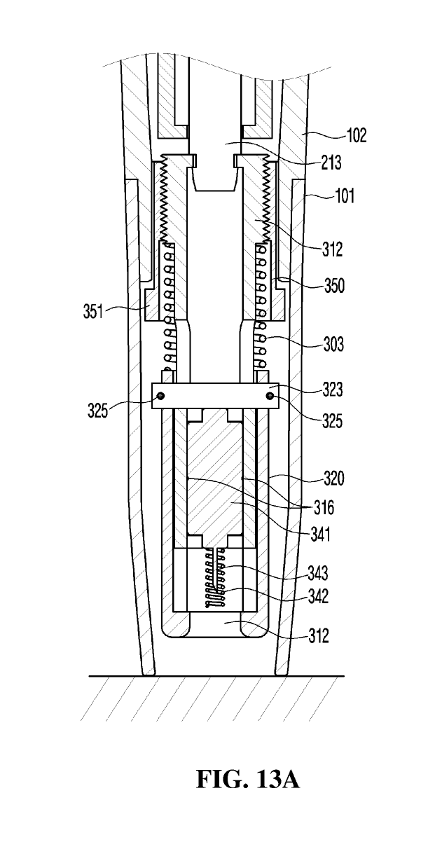

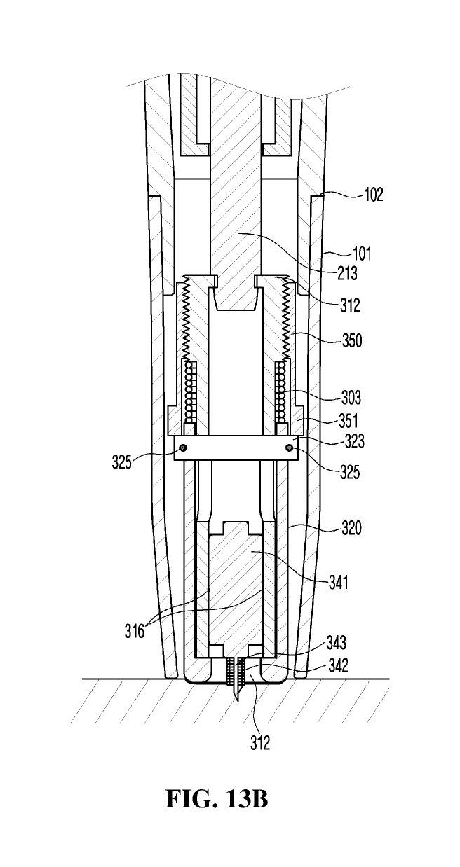

FIGS. 1-13 are views of the lancing device according to a first embodiment of the present invention.

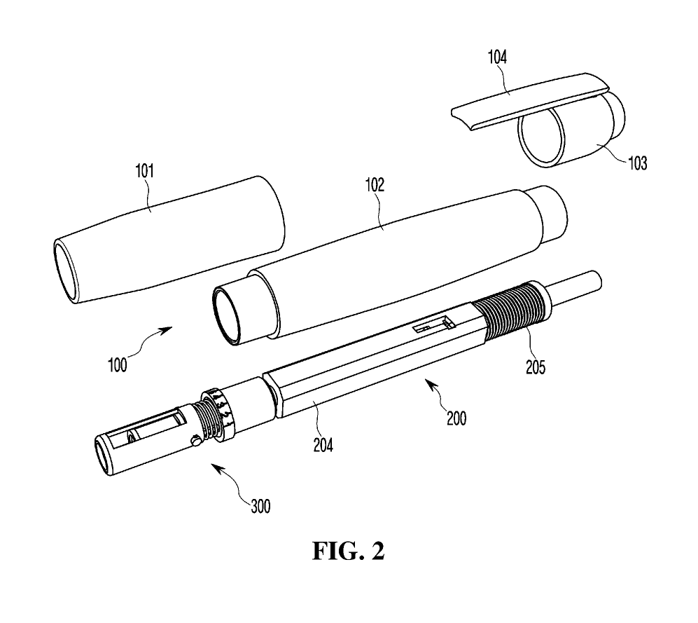

As shown in FIGS. 1 and 2, the lancing device according to the first embodiment of the present invention includes: a casing 100 that forms an outer appearance of the device; and a launch part 200 for launching a bumper part 300 having a bumper 320 and a needle body 341 to strike the skin, in which the bumper part 300 has a needle body holder 310 including the bumper 320 that primarily strikes the skin and a needle spring 342 that strikes the skin secondarily.

When the bumper part 300 is launched, the bumper part 300 first strikes the skin by the inertial force at the time of launching, and then continues to press the skin while being in contact with the skin by the compressive force of a bump spring 303. At this time, the bump spring 303 is compressed due to the inertial force of the bumper 320, which is momentarily maintained, and the inertial force of the needle body holder 310 including the lancet. A stop bar 323 seated on an upper end of the bumper 320 then moves up to a tip of an adjusting member flange 351 to be properly adjusted along a rectangular hole 313 of a cylindrical body 312, and the needle spring 342 of the needle body 341 strikes the skin. Immediately thereafter, the needle 343 penetrates into the subcutaneous tissue of the skin, and then the entire bumper part 300, that is, the bumper 320, the needle body holder 310, a depth adjusting member 350, the stop bar 323, a cotter pin 325, the needle body 341, and the needle spring 342 are simultaneously returned to the initial position before the launch by a bumper return spring 214. At this time, a penetration depth of the needle 343 can be controlled by the vertical movement of the stop bar 323 only to the extent of the adjustment position set by the adjusting member flange 351.

FIG. 1 is a perspective view of a lancing device according to the first embodiment of the present invention, showing the casing 100. As shown in FIGS. 1 and 2, the casing 100 includes a cylindrical front cap 101, a sleeve 102 having one side coupled to the front cap 101 and the other side coupled to a rear cap 103, and the rear cap 103 coupled to the sleeve 102. The front cap 101, the sleeve 102 and the rear cap 103 are preferably screw-coupled together. The rear cap 103 may be provided with a clip 104 for convenient carrying.

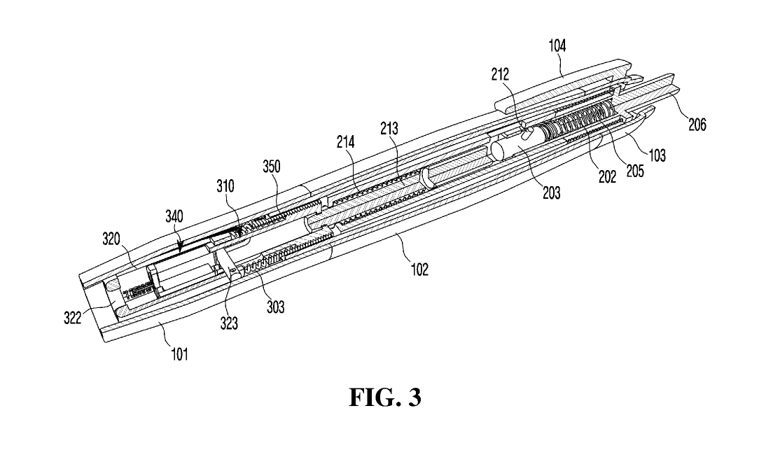

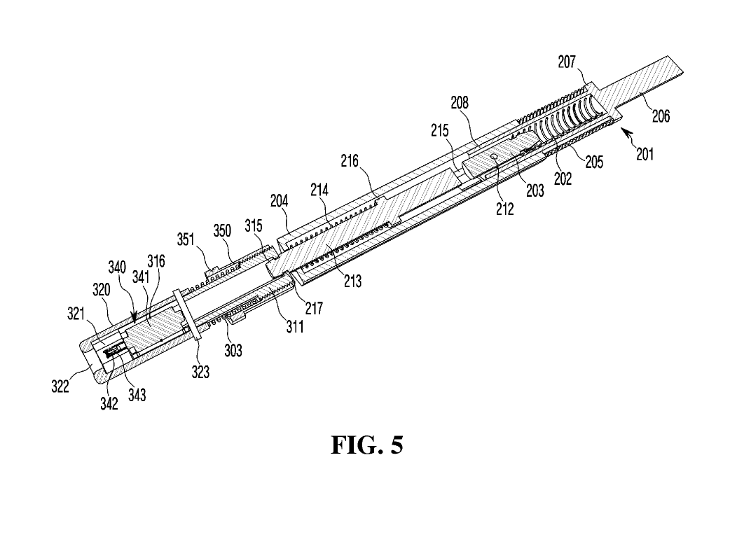

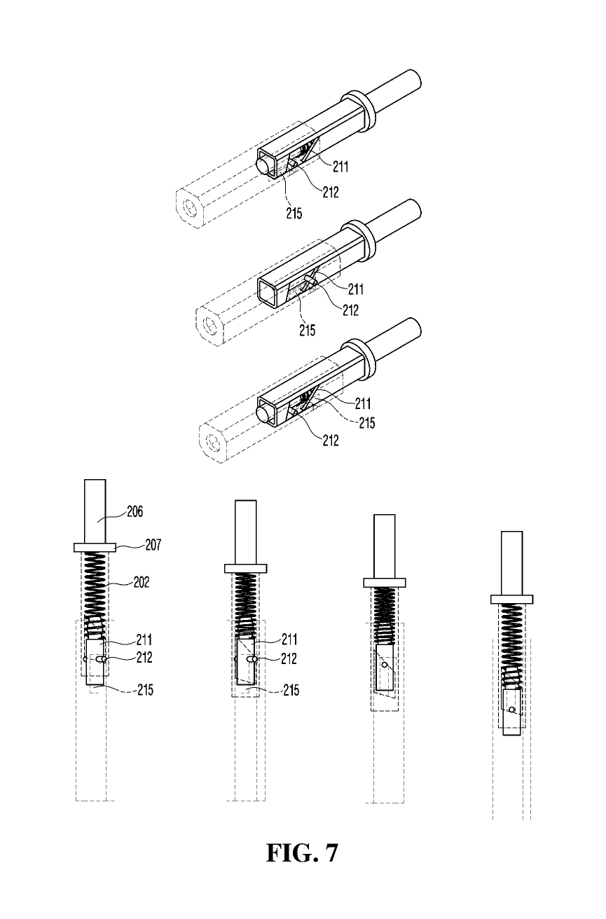

As shown in FIGS. 3-7, the launch part 200 includes a launch body 201, a launch spring 202, a launch pestle 203, a square tube 204 and a launch body return spring 205. As shown in detail in FIGS. 5-7, the launch body 201 integrally comprises a push member 206, a launch flange 207 formed at the tip of the push member 206, and a hollow tetrahedron 208 which is open at one side to accommodate the launch pestle 203 and the launch spring 202. As shown in detail in FIG. 7, in a lower part of the hollow tetrahedron 208, there are formed square holes 211 whose upper sides are sloped on one surface and the opposite surface, respectively. The upper sides of the two square holes 211 are opposite to each other (i.e., ‘V or’/′). A launch spring 210 is inserted into one side of the launch pestle 203 and a trigger pin 212 is coupled through a middle part of the launch pestle 203.

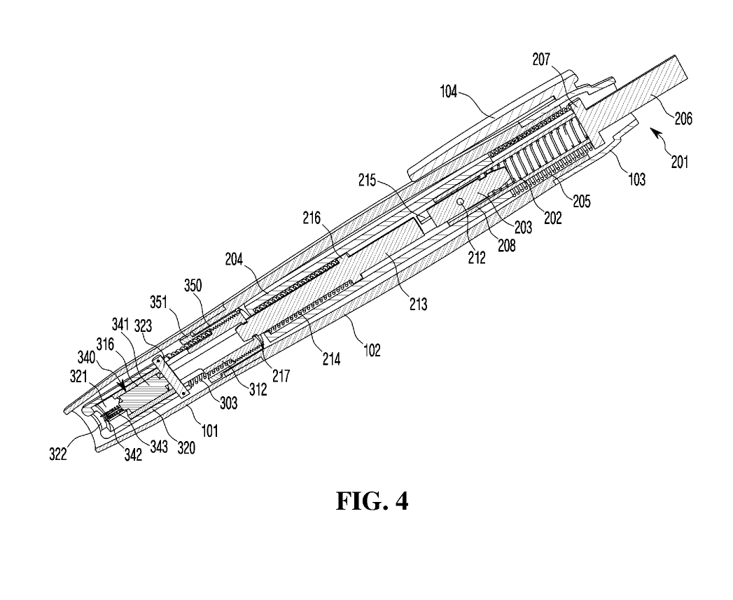

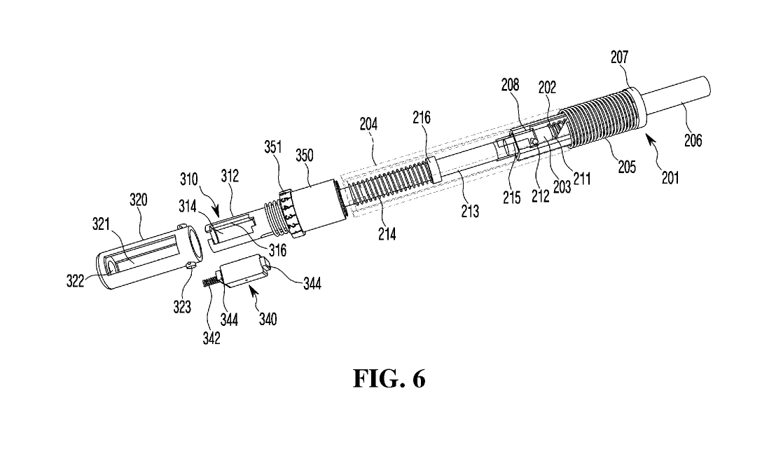

As shown in detail in FIGS. 4-6, a rectangular hole (not shown) is formed in the upper part of the square tube 204 to insert the hollow tetrahedron 208 of the launch body 201, and a circular hole (not shown) is formed in the lower part of the square tube 204 to insert a columnar coupling shaft 213. As shown in detail in FIGS. 6 and 7, the square tube 204 is configured to receive the coupling shaft 213, the bumper return spring 214 and half of the tetrahedron 208 of the launch body 201. Two ‘1’-shaped slits 215 are formed on one side and the opposite side of the square tube 204, respectively, and the two slits 215 are formed asymmetrically. That is, one of the two transverse slits 215 has a ‘1’ shape and the other has a ‘F’ shape. The slits 215 are fitted with a trigger pin 212 to move.

A launch body return spring 205 for returning the launch body 201 is interposed between the upper end of the square tube 204 and the launch flange 207.

Referring to FIG. 7, the operation of the launch part 200 will be described as follows.

Before launching, the trigger pin 212 is positioned at the lower side of the square hole 211 and in the transverse slits of the ‘1’-shaped slits 215. The push member 206 is pressed by about 10 mm to launch the bumper 320 and the needle holder 302, then the launch spring 210 connected to the launch pestle 203 in the hollow tetrahedron 208 is compressed. The trigger pin 212 passing through the launch pestle 203 connected to the launch spring 210 is slid along the upper side of the rectangular hole 211 and is “twisted”, and then is pushed along the transverse slits of the ‘1’-shaped slits 215 and momentarily falls below the longitudinal slits of the ‘1’-shaped slits 215, whereby the launch pestle 203 can be launched. When the launch pestle 203 is lowered to the end of the transverse slits of the ‘1’-shaped slits, a launching force is transmitted to the bumper part 300 through the coupling shaft 213, so that the bumper 320 strikes the skin. When the push member 206 is pushed again, the launch body 201 and the launch pestle 203 return to the transverse slits of the ‘1’-shaped slits 215 and return to the standing state.

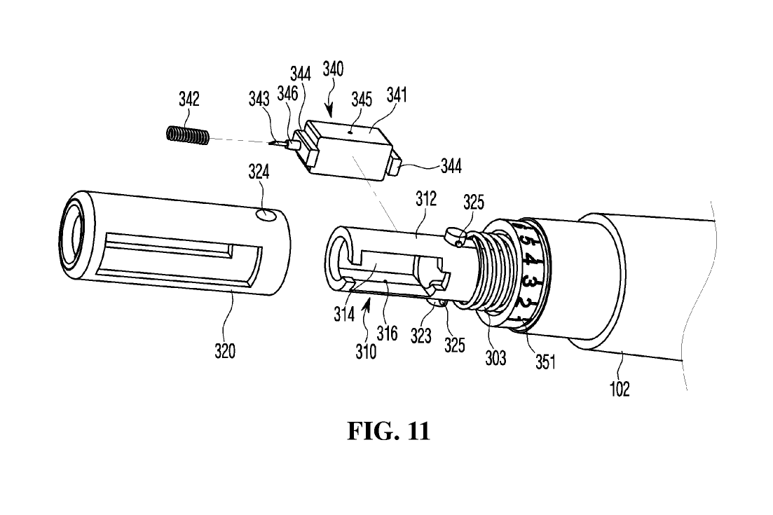

As shown in FIGS. 6 and 11, the bumper part 300 mainly includes the needle body holder 310, the bumper 320, and the bump spring 303.

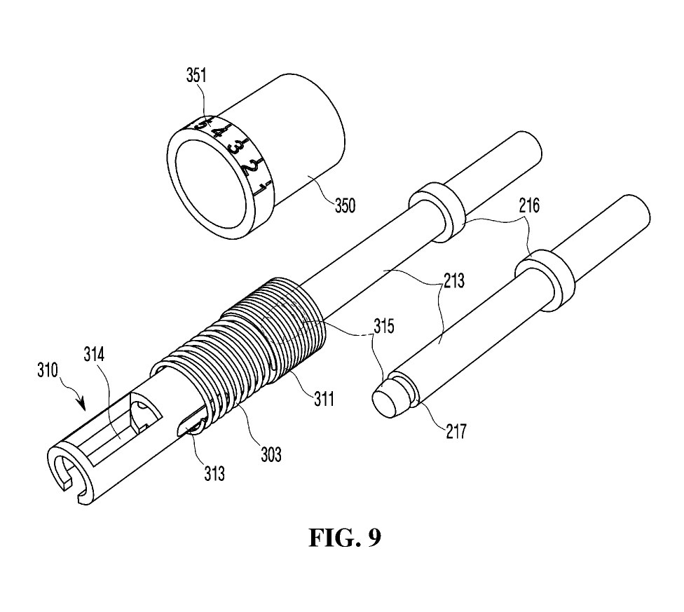

As shown in detail in FIG. 9, the needle body holder 310 includes a holder flange 311 formed at its upper portion and a cylindrical body 312 formed under the holder flange 311. A rectangular hole 313 is formed in the cylindrical body 312 so that the stop bar 323 moves up and down. A needle body insertion hole 314 for inserting a lancet 340 is formed in the lower portion of the cylindrical body 312.

As shown in FIG. 11, one side of the needle body insertion hole 314 is opened to allow the needle body 341 to be inserted and removed laterally, and the two inner walls of the needle body insertion hole 314 are configured to be flat to engage with the two lateral surfaces of the needle body 341. The lower end of the cylindrical body 312 is opened to allow the needle 343 of the lancet 340 and the needle spring 342 to move in and out. A needle body fixing protrusion 316 fixed to the fixing groove 345 of the needle body 341 is formed on the inner walls of the needle body insertion hole 314. As shown in FIG. 9, a ring-shaped protrusion 315 is formed on the upper end of the holder flange 311 toward the inside of the holder flange 311 so as to be coupled to a circular-shaped coupling groove 217 formed along the outer peripheral surface of the lower end of the coupling shaft 213. A screw thread for engaging with a spiral formed on the inner side of the depth adjusting member 350 is formed on the outer peripheral surface of the holder flange 311.

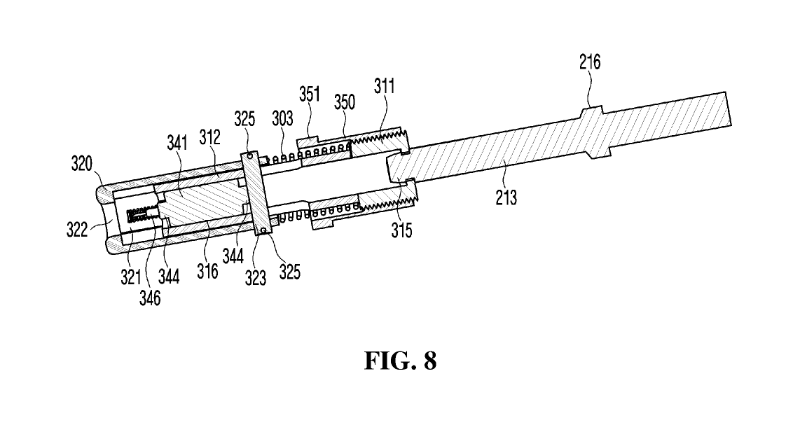

As shown in FIGS. 8 and 9, the coupling shaft 213 has a columnar shape and has a coupling shaft flange 216 for inserting and fixing the return spring 214. The coupling groove 217 is formed at the lower end of the coupling shaft flange 216 so as to be engaged with the ring-shaped protrusion 315 formed toward the inside of the holder flange 311.

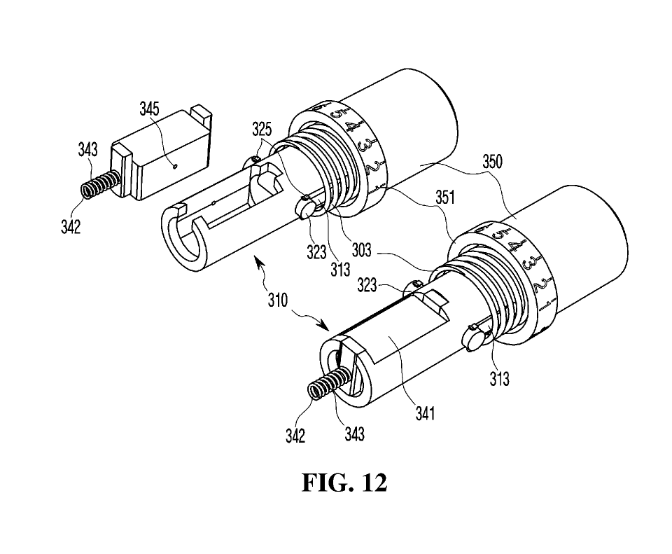

As shown in FIGS. 11 and 12, a needle body fixing protrusion 316 for fixing the needle body 341 is formed on the inner walls of the needle body insertion hole 314. The needle body fixing protrusion 316 is preferably in the form of a bead of a metal or plastic material.

As shown in detail in FIGS. 6 and 11, the bumper 320 is cylindrical, and the upper part of the bumper 320 is open to receive the needle body holder 310 and has a rectangular hole 321 for inserting and removing the lancet 340 in the longitudinal direction. A needle access hole 322 is formed at the lower end of the bumper 320 to allow the needle 343 and the needle spring 342 to move in and out. A stop bar insertion port 324 is formed in the upper end of the bumper 320 to insert the stop bar 323. As shown in detail in FIGS. 8-11, the stop bar 323 is inserted through the rectangular hole 313 and the stop bar insertion port 324 of the needle body holder 310. The cotter pins 325 are fitted at both ends of the stop bar 323 so that the stop bar 323 is not separated from the bumper 320.

As shown in detail in FIGS. 5, 8, 10 and 13, the bump spring 303 is inserted between the lower surface of the holder flange 311 of the needle body holder 310 and the upper surface of the bumper 320 while surrounding the cylindrical body 312.

As shown in detail in FIGS. 11-13, the lancet 340 inserted into the lancing device according to the first embodiment of the present invention includes the needle body 341, the needle spring 342, and the needle 343. The needle body 341 has protrusions 344 formed at both ends along the longitudinal direction of the needle body 341 and is configured to be inserted in conformity with the shape of the needle body insertion hole 314. The needle 343 is embedded in the needle body 341. Fixing grooves 345 are formed on both sides of the needle body 341 so as to be fastened to the needle body fixing protrusions 316 provided on the inner wall of both sides of the needle body insertion hole 314 of the needle body holder 310.

The lancet 340 according to the first embodiment of the present invention is disposable and the needle spring 342 completely surrounds the needle 343 to prevent secondary infections such as hepatitis and AIDS caused by careless handling.

The lancing device according to the first embodiment of the present invention may be provided with a depth adjusting member 350 to adjust the needle penetration depth since people's skin thickness is different. As shown in detail in FIGS. 4, 5, and 13, the depth adjusting member 350 is cylindrical and is open at its upper and lower ends. A spiral for coupling with a screw formed on the holder flange 311 of the needle body holder 310 is formed on the upper inner circumferential surface of the depth adjusting member 350 and an adjusting member flange 351 is formed at a lower end thereof.

As shown in detail in FIGS. 3 and 4, the adjusting member flange 351 is engaged with the screw formed in the holder flange 311 at the top of the cylindrical body 312 of the needle body holder 310, and the penetration depth of the needle 343 can be adjusted by turning the depth adjusting member 350 to the left or right. When the adjusting member flange 351 is turned to the right, the needle 343 penetrates deeper into the skin. At this time, the stop bar 323 moves along the rectangular hole 313 and the movement distance becomes longer, so that the skin penetrates deeper into the skin. On the contrary, when the adjusting member flange 351 is turned to the left, the needle 343 penetrates the skin more shallowly. At this time, the stop bar 323 moves along the rectangular hole 313 and the movement distance becomes shorter, so that the skin penetrates the skin more shallowly.

Referring to FIGS. 3-5, 8, 11 and 13, the operation of the bumper part 300 of the lancing device according to the first embodiment of the present invention will be described below.

When the push member 206 of the launch part 200 is pushed to trigger the launch pestle 203 to strike the coupling shaft 213, a force is transmitted to the needle body holder 310 through the ring-shaped protrusion 315 formed toward the inner side of the coupling groove 217 of the coupling shaft 213 and the holder flange 311 coupled to the coupling groove 217. At this time, the bump spring 303 should be strong enough to transmit the launching force to the bumper 320. The lower end surface of the bumper 320 strikes the skin due to the launching force, then the bump spring 303 is compressed and the needle spring 342 of the needle body 341 inserted into the needle body holder 310 strikes the skin. Immediately thereafter, the needle 343 penetrates the subcutaneous tissue of the skin, and then the entire bumper part 300, that is, the bumper 320, the needle spring 342 and the needle 343 are simultaneously returned by the return spring 214. While the bumper 320 is striking the skin and contacting the skin, the needle spring 342 inserted into the needle body holder 310 strikes the skin, and then the needle 343 penetrates the skin immediately when the needle spring 342 is in contact with the skin. At the same time, the stop bar 323 rises along the rectangular hole 313 of the needle body holder 310 and stops to the lower end of the adjusting member flange 351. This point is the limit that the needle 343 penetrates the skin. At this time, the depth of penetration of the needle 343 is adjusted by causing the stop bar 323 to perform a vertical movement only to the end of the screw adjusting position of the adjusting member flange 351. After the bumper 320 strikes the skin, the needle spring 342 strikes the skin again in contact with the skin. Further, after the needle 343 pierces the skin while the bumper 320 and the needle spring 342 are in contact with the skin, the bumper 320, the needle spring 342, and the needle 343 are simultaneously released from the skin by the bumper return spring 214. The needle spring 342 extends a little longer than the needle 343 and is inserted or fixed to the needle fixing protrusion 346 formed on the protrusion 344 while surrounding the needle 343.

As described above, since the bumper 320 first strikes the skin, and then the needle spring 342 strikes the skin secondarily with a time difference of about 0.1 second, the needle 343 pierces the skin almost at the same time, and the subject will not feel any pain at all.

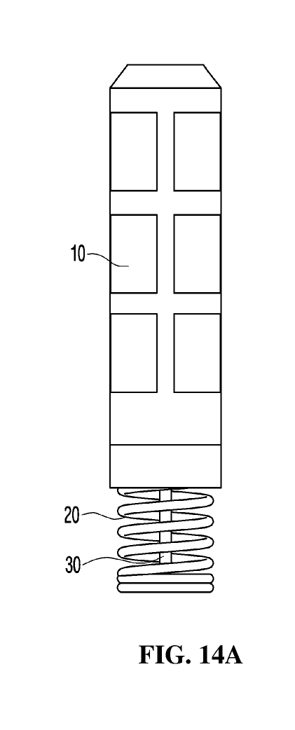

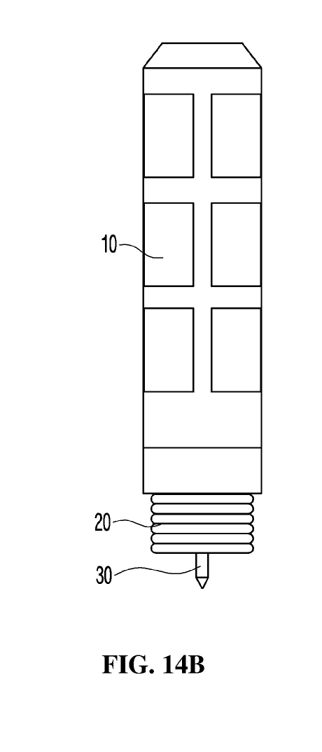

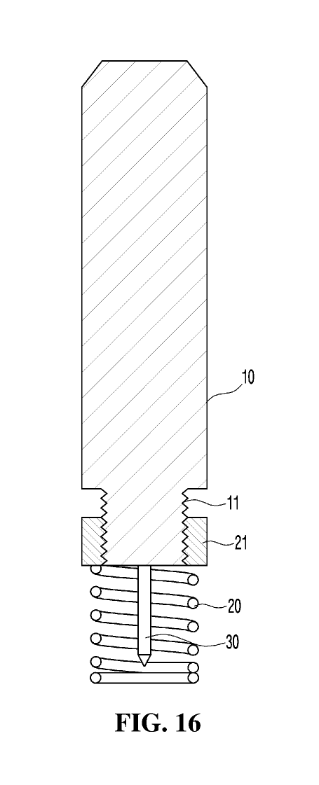

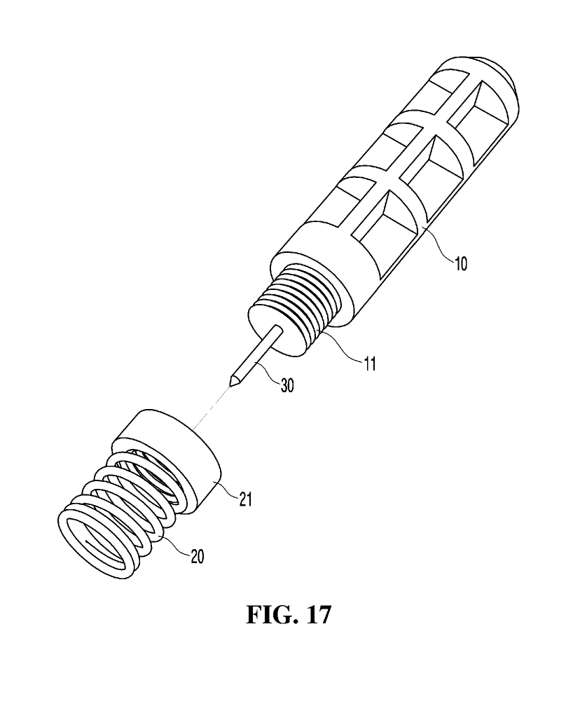

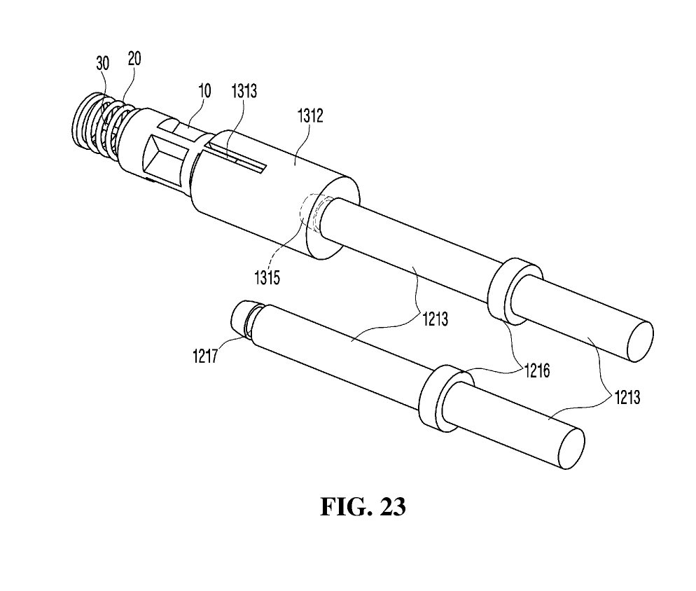

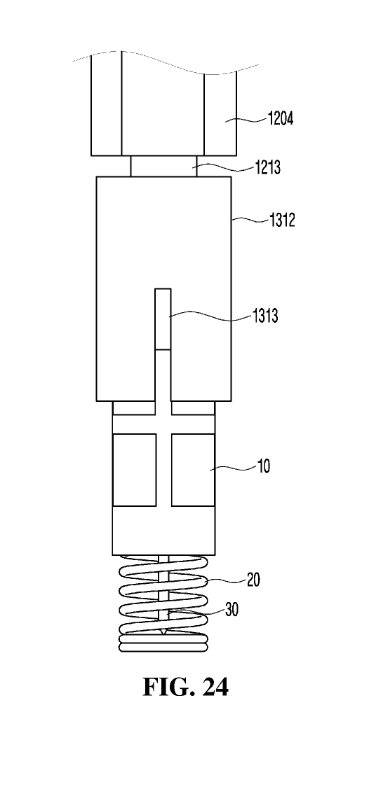

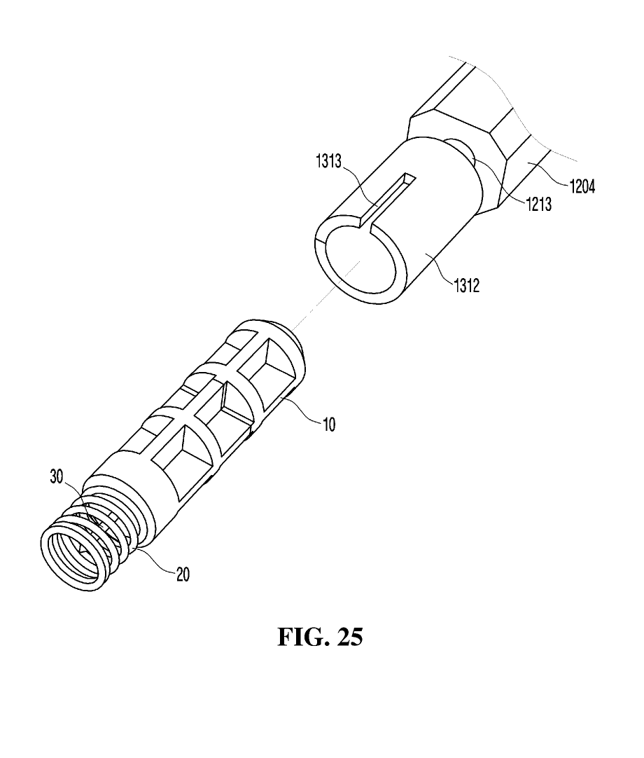

FIGS. 14-26 are drawings of a disposable painless lancet and a lancing device according to a second embodiment of the present invention.

Hereinafter, the disposable painless lancet and the lancing device for therewith according to the second embodiment of the present invention will be described.

As shown in FIG. 14, the disposable painless lancet according to the second embodiment of the present invention includes a needle body 10, a needle 30 embedded in the center of one end of the needle body 10 and a bump spring 20 embedded in one end of the needle body 10 to surround the needle 30.

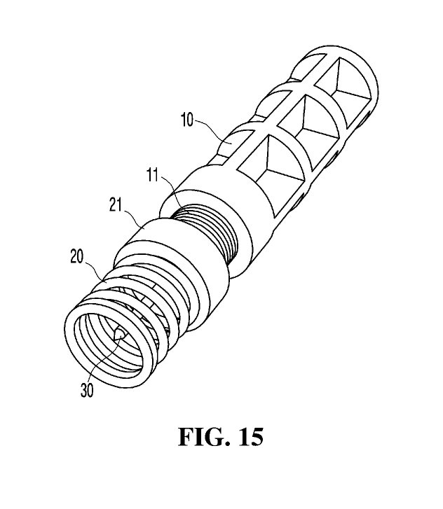

The material of the needle body 10 is preferably plastics. The needle body 10 may have a columnar structure, preferably a skeleton structure as shown in FIGS. 14 and 15.

The needle 30 is embedded or fixed at the center of one end of the needle body 10. As shown in FIG. 14 (a), a part of the bump spring 20 is embedded or fixed to the center of one end of the needle body 10 so as to surround the needle 30 longer than the length of the needle 30.

The length of the needle 30 can be standardized in the production of the product. In the drawings attached to the present specification, the tip of the needle 30 is conical, but may be angular.

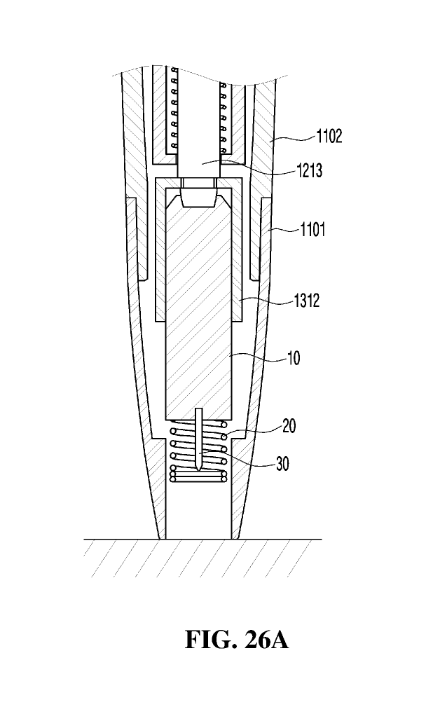

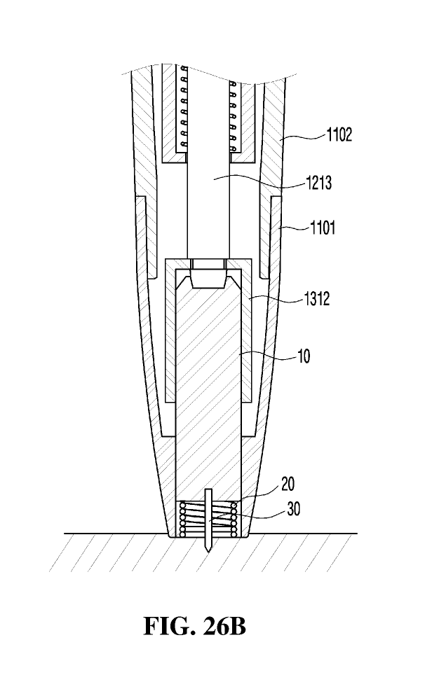

FIG. 14 (b) shows the state of the disposable painless lancet when contacted with the skin (40 in FIG. 26) after being launched from the lancing device for sampling blood. When the disposable painless lancet of the present invention is launched from the lancing device, the bump spring 20 momentarily strikes a skin 40. At this time, the skin 40 is first impacted by the momentary striking applied by the bump spring 20, disturbing the skin nerve not to feel the pain. After the needle 30 has pierced the skin 40, the needle 30 is momentarily released out of the skin 40 due to the force of restoration of the bump spring 20 and the time the needle 30 stays on the skin is very short. Also, the needle 30 does not penetrate excessively below the subcutaneous tissue. The bump spring 20 is compressed by inertia at the time of launching and the needle 30 penetrates the skin 40 while pressing the skin 40 harder and harder so that the pain is reduced or eliminated when the needle 30 is pierced. This is like the principle that a nurse in a hospital pierces a needle immediately after striking the skin with the palm of a hand to relieve pain.

As shown in FIG. 26, since the bump spring 20 determines a limit of a length that can be pressed immediately after striking the skin 40 and the needle 30 can penetrate the skin 40, the needle 30 penetrates the skin 40 only by a predetermined length. Therefore, it is possible to prevent the needle 30 from being excessively penetrated into the skin 40 more than necessary for sampling blood.

Also, after the needle 30 has penetrated the skin, the blood sticks to the needle 30 inevitably. However, since the needle 30 is hidden in the bump spring 20, there is no fear that the blood stuck on the needle 30 comes into contact with a person, thereby preventing secondary infections of various diseases such as AIDS and hepatitis.

FIGS. 15-17 are views showing a disposable painless lancet having a spiral part 11 and a spring body 21, which are means for adjusting the depth of the needle 30 into the skin 40 according to the second embodiment of the present invention.

One end of the needle body 10 has the columnar spiral part 11 smaller than the diameter of the needle body 10 and one end of the needle 30 is embedded in the center of the end of the spiral part 11. The spring body 21 is nut-shaped and is screwed onto the spiral part 11. A spiral is formed inside the spring body 21, and one end of the bump spring 20 is embedded or fixed to one side of the spring body 21. When the spring body 21 is turned clockwise, the needle 30 penetrates the skin 40 deeper, and when the spring body 21 is turned counterclockwise, the needle 30 penetrates the skin 40 more shallowly.

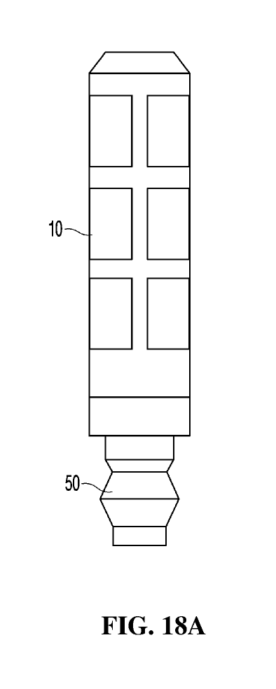

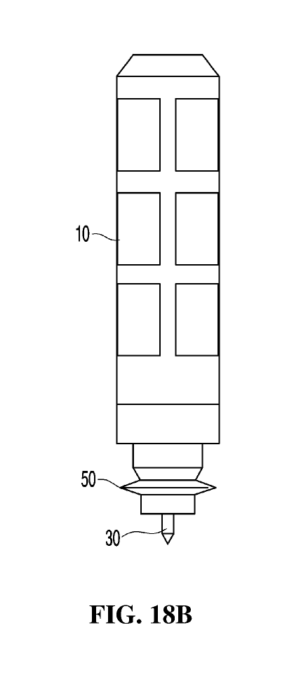

As shown in FIG. 18, in the present invention, instead of the bump spring 20, a silicone or elastic member 50 may be used for a spring function.

In the lancing device according to the second embodiment of the present invention, the principle of operation of the disposable painless lancet in which the silicone or elastic member 50 is used in place of the bump spring 20 is the same as that of the lancet using the bump spring 20.

Further, the present invention provides a lancing device for use with the disposable painless lancet according to the second embodiment of the present invention.

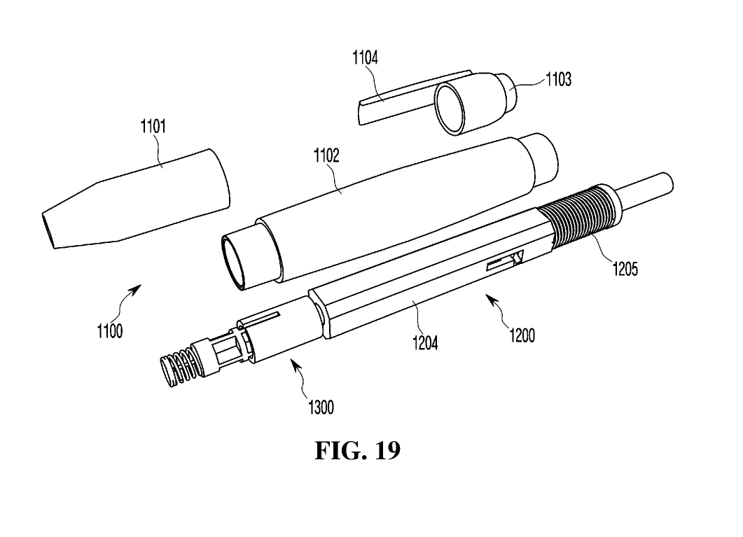

As shown in FIG. 19, the lancing device according to the second embodiment of the present invention mainly includes a casing 1100 forming an outer appearance thereof, a launch part 1200 for launching the needle body 10 in a needle body holder 1300, in which the needle body holder 1300 is configured for holding and fixing the needle body 10.

As shown in FIG. 19, the casing 1100 includes a front cap 1101, a sleeve 1102 having one end coupled to the front cap 1101 and the other end coupled to the rear cap 1103, in which the rear cap 1103 is coupled to the sleeve 1102. The front cap 1101, the sleeve 1102, and the rear cap 1103 are preferably screwed together. The rear cap 1103 may be provided with a clip 1104 for convenient carrying.

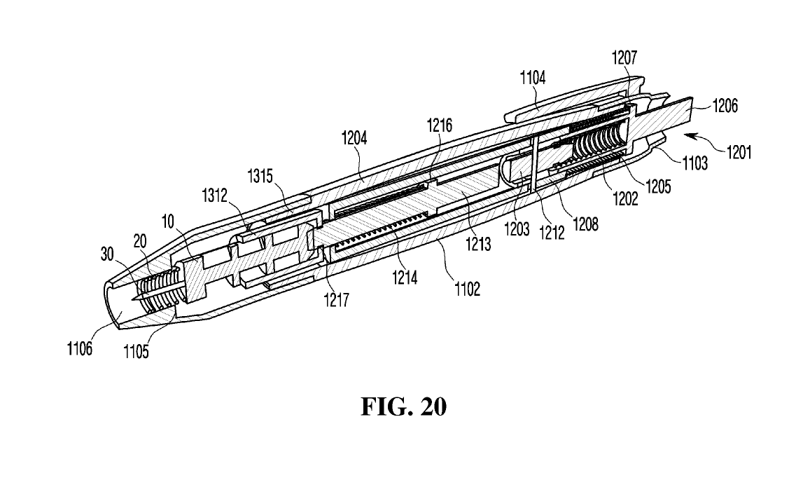

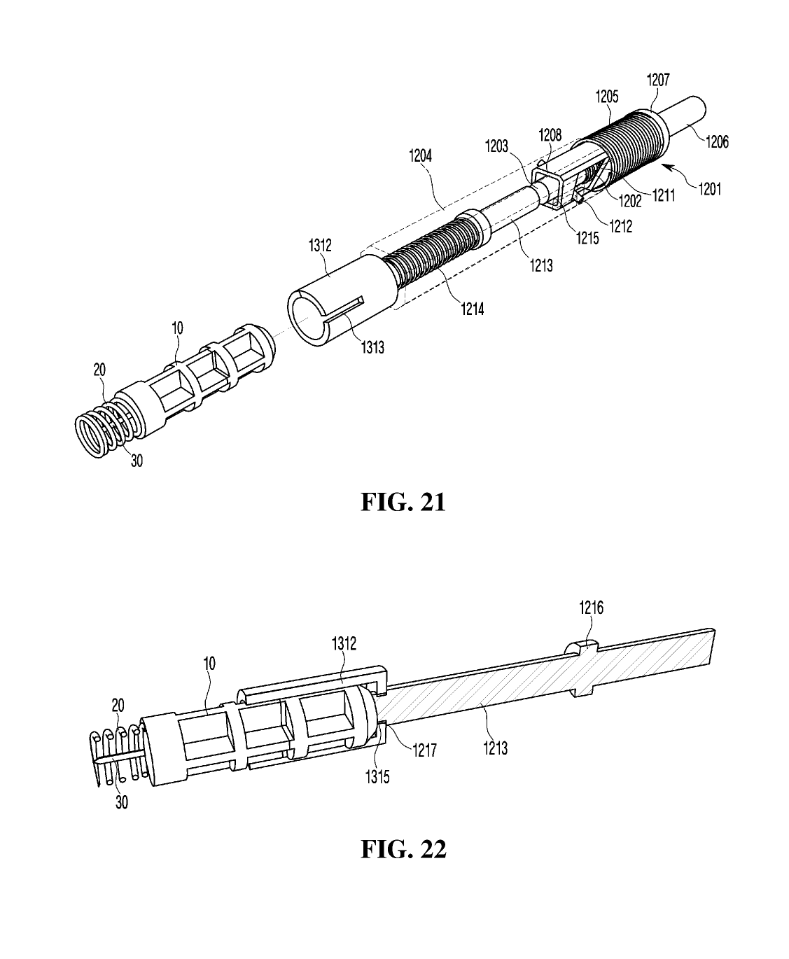

As shown in FIGS. 20 and 21, the launch part 1200 includes a launch body 1201, a launch spring 1202, a launch pestle 1203, a square tube 1204, and a launch body return spring 1205. As shown in detail in FIGS. 19-21, the launch body 1201 integrally comprises a push member 1206, a launch flange 1207 formed at the tip of the push member 1206, and a hollow tetrahedron 1208 which is open at one side to accommodate the launch pestle 1203 and the launch spring 1202. As shown in detail in FIG. 21, in the lower part of the hollow tetrahedron 1208, there are formed square holes 1211 whose upper sides are sloped on one surface and the opposite surface, respectively. The upper sides of the two square holes 1211 are opposite to each other i.e., ‘\’ or ‘/’). A launch spring 1210 is inserted into one side of the launch pestle 1203 and a trigger pin 1212 is coupled through the middle part of the launch pestle 1203.

In the lancing device according to the second embodiment of the present invention, the structure and operation principle of the launch part 1200 are as described above with reference to FIGS. 3-7 of the lancing device according to the first embodiment of the present invention.

As shown in detail in FIGS. 20 and 21, a ring-shaped protrusion 1315 is formed in the upper position of the needle body holder 1300 so as to be engaged with a coupling groove 1217 of a coupling shaft 1213. The needle body holder 1300 is open at its lower portion and has one incision groove 1313 cut from the lower portion to the middle portion thereof. The incision groove 1313 is configured to elastically hold the needle body 10 firmly.

As shown in FIG. 20, the front cap 1101 of the casing 1100 has a shape gradually tapering from the top to the bottom. A circular engagement protrusion 1105 is formed on the inside of the front cap 1101 to limit the downward movement of the lower surface of a cylindrical body 1312. A guide hole 1106 is formed from the engagement protrusion 1105 to the lower end of the front cap 1101 to guide the needle body 10 and the bump spring 20 in a predetermined direction.

Referring to FIGS. 20 and 21, the operation of the needle body holder 1300 of the lancing device and the disposable painless lancet according to the second embodiment of the present invention will be described below.

When the push member 1206 of the launch part 1200 is pushed to trigger the launch pestle 1203 to strike the coupling shaft 1213, a force is transmitted to the needle body holder 1310 through the ring-shaped protrusion 1315 formed toward the inner side of the coupling groove 1217 of the coupling shaft 1213 and the cylindrical body 1312 coupled to the coupling groove 1217. The force is then transmitted to the lancet including the needle body 10 inserted in the cylindrical body 1312, the bump spring 20 and the needle 30. Then, the needle body 10 of the lancet and the bump spring 20 are guided through the guide hole 1106 and then the bump spring 20 strikes the skin. The needle 30 penetrates into the capillary blood vessels of the skin while the bump spring 20 is compressed, and then the cylindrical body 1312 of the needle body holder 1300 and the whole lancet are simultaneously returned to the position before the launching by the bump spring 20.

As described above, since the bump spring 20 strikes the skin to disturb the skin nerves, and then the needle 30 pierces the skin with a slight difference in time, and the time during which the needle 30 stays in the skin 40 is significantly shortened by the restoring force of the bump spring 20, thereby completely eliminating the pain.

According to the second embodiment of the present invention, when the disposable painless lancet having the silicone or elastic member 50 in place of the bump spring 20 is inserted in the lancing device, the operation principle of the lancing device is the same as the principle described in connection with the bump spring 20.

The present inventors conducted an experiment for painlessness by use of the lancing device according to the present invention in 50 adult males (mean age 42.5 years). The disposable painless lancets were launched on the skin of their ring fingers. Blood flowed a little, but all 50 people could not feel the pain.

1. A painless lancing device comprising:
a casing 100 including:
a cylindrical front cap 101;
a sleeve 102 connected to the cylindrical front cap 101 at a lower end; and
a rear cap 103 connected to an upper end of the sleeve 102;
a launch part 200 disposed in the casing 100 configured to launch a cylindrical bumper 320 and a needle body 341 to strike a skin; and
a bumper part 300 disposed in the casing 100 and connected to the launch part 200 by a coupling shaft 213,
wherein the bumper part 300 comprises a needle body holder 310 including:
a holder flange 311 disposed at an upper portion thereof;
a first rectangular hole 313 disposed below the holder flange 311 so that a stop bar 323 moves up and down; and
a needle body insertion hole 314 disposed at a lower portion of the bumper part 300 to insert a lancet 340;
wherein the cylindrical bumper 320 is configured to receive the needle body holder 310 at an upper end thereof, the cylindrical bumper 320 comprising:
a second rectangular hole 321 disposed along a longitudinal direction to insert and remove the lancet 340;
a needle access hole 322 disposed at a lower end of the cylindrical bumper 320 for a needle 343 and a needle spring 342 to move in and out therethrough; and
a stop bar insertion port 324 disposed at the upper end to insert the stop bar 323; and
a bump spring 303 surrounding a cylindrical body 312 and disposed between a lower portion of the holder flange 311 of the needle body holder 310 and an upper portion of the cylindrical bumper 320.

2. The painless lancing device according to claim 1, wherein one side of the needle body insertion hole 314 is configured to allow insertion and removal of the needle body 341 from said one side, and the needle body 341 is disposed to align and fit within the needle body insertion hole 314.

3. The painless lancing device according to claim 1, wherein the cylindrical body 312 has a lower end configured to allow the needle 343 of the lancet 340 and the needle spring 342 to move in and out,
wherein a ring-shaped protrusion 315 is disposed at an inside surface of an upper end of the holder flange 311 so that the ring-shaped protrusion 315 is engaged with a circular-shaped coupling groove 217 disposed along a lower outer peripheral surface of the coupling shaft 213, and
wherein the holder flange 311 has a screw thread disposed on an outer circumferential surface thereof for screw coupling with a spiral disposed on an inside surface of a depth adjusting member 350.

4. The painless lancing device according to claim 1, wherein inner surfaces of the needle body holder 310 have a plurality of needle body fixing protrusions 316 for fixing the needle body 341.

5. The painless lancing device according to claim 4, wherein the lancet 340 further comprises:
a rectangular needle body 341;
an upper protrusion and a lower protrusion 344 disposed on an upper side and a lower side of the needle body 341, respectively, wherein a needle fixing protrusion is disposed on the lower protrusion 346, the needle spring 342 is inserted and fixed on the needle fixing protrusion 346, and the needle 343 is inserted into the needle body 341 through the needle fixing protrusion 346; and
a plurality of fixing grooves 345 to be fixed on the plurality of needle body fixing protrusions 316 provided on the inner surfaces of the needle body holder 310.

6. The painless lancing device according to claim 1, further comprising a depth adjusting member 350 configured to adjust a depth of skin penetration of the needle 343 and having an open upper end and an open lower end, the depth adjusting member comprising:
a spiral disposed in an inner surface thereof for engaging with a screw thread disposed on an outer circumferential surface of the holder flange 311 of the cylindrical body 312 of the needle body holder 310; and
an adjusting member flange 351 disposed at a lower end of the depth adjusting member 350.

7. The painless lancing device according to claim 1, wherein the bumper part 300 is connected to the coupling shaft 213 by engaging a ring-shaped protrusion 315 disposed at an inside surface of an upper end of the holder flange 311 with a circular-shaped coupling groove 217 disposed along a lower outer peripheral surface of the coupling shaft 213, the coupling shaft 213 comprising a coupling shaft flange 216 for inserting a return spring 214 therein.

8. A disposable painless lancet comprising:
a needle body 10;
a needle 30 embedded at a center of one end of the needle body 10; and
a bump spring 20 embedded or fixed at said one end of the needle body 10 so as to surround the needle 30 and extend beyond the needle 30.

9. The disposable painless lancet according to claim 8,
wherein the needle body 10 comprises an elongated spiral part 11 disposed at one end thereof; and an end of the needle 30 is embedded in a center of an end of the elongated spiral part 11,
wherein a spring body 21 having a screw thread disposed in an inner surface thereof is screw coupled with the elongated spiral part 11, and
wherein one end of the bump spring 20 is embedded or fixed to the spring body 21.

10. A disposable painless lancet, comprising:
a needle body 10;
a needle 30 embedded at a center of one end of the needle body 10; and
an elastic member 50 embedded or fixed at said one end of the needle body 10 so as to surround the needle 30 and extend beyond the needle 30.

11. A painless lancing device, comprising:
a casing 1100 including:
a cylindrical front cap 1101;
a sleeve 1102 connected to the cylindrical front cap 1101 at a bottom end; and
a rear cap 1103 connected to a top end of the sleeve 1102;
a launch part 1200 for launching a needle body 10 disposed inside a cylindrical body 1312; and
a needle body holder 1300 for holding and fixing the needle body 10,
wherein the cylindrical front cap 1101 has a gradually tapering shape from a top end to a bottom end, and the cylindrical front cap 1101 comprises:
a circular engagement protrusion 1105 disposed on an inside surface of the cylindrical front cap 1101 to limit a downward movement of a bottom surface of the cylindrical body 1312; and
a guide hole 1106 provided from the circular engagement protrusion 1105 to the bottom end of the cylindrical front cap 1101 to guide the needle body 10 and a bump spring 20 in a predetermined direction,
wherein the needle body holder 1300 comprises a ring-shaped protrusion 1315 disposed in an inside surface of the needle body holder 1300 so as to be engaged with a coupling groove 1217 of a coupling shaft 1213, and
wherein the needle body holder 1300 is open at a bottom portion and has one incision groove 1313 on its side provided from the lower portion to a middle portion thereof.

12. The disposable painless lancet of claim 10, wherein the elastic member 50 is a silicone.

13. The disposable painless lancet of claim 10,
wherein the needle body 10 comprises an elongated spiral part 11 disposed at one end thereof; and an end of the needle 30 is embedded in a center of an end of the elongated spiral part 11,
wherein a spring body 21 having a screw thread disposed in an inner surface thereof is screw coupled with the elongated spiral part 11, and
wherein one end of the elastic member 50 is embedded or fixed to the spring body 21.

14. The disposable painless lancet of claim 13, wherein the elastic member 50 is a silicone.
